SEQUENCE LISTING (34 sequences)

SEQ ID NO: 1 - protein, 117 aa, Mus musculus
EVQLQQSGPELVKPGASVKISCKTSGHTFTEYNMQWVKQSLGQSLEWIGGINPNNVGSIYNQKFRGKATLTVDKSSSTAYMELRSLTSEDSAVYYCARGYGNYVAYWGQGTLVTVSA

SEQ ID NO: 2 - protein, 107 aa, Mus musculus
DIVMTQSQKFMSTSVGDRVSVTCKASQNVNTNVAWYQQKPGQSPKSLIYSASYRYSGVPDRFTGSGSGTDFTLTISNVQSEDLAEFFCQQYNRYPFTFGGGTKLELK

SEQ ID NO: 3 - protein, 645 aa, Homo sapiens
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGT

SEQ ID NO: 4 - protein, 347 aa, Homo sapiens
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 5 - protein, 17 aa, Mus musculus
GINPNNVGSIYNQKFRG

SEQ ID NO: 6 - protein, 8 aa, Mus musculus
GYGNYVAY

SEQ ID NO: 7 - protein, 10 aa, Homo sapiens
TLLSVTRNDV

SEQ ID NO: 8 - protein, 9 aa, Homo sapiens
YLSGANLNL

SEQ ID NO: 9 - protein, 117 aa, Artificial Sequence
  modified heavy chain variable region of murine antibody
EVQLQQSGPETGKPGASGKMSCKTSGHTSTEHNGQWAKQSPGQSLEWIGGINPNNVGSIYNQKFRGKATLTADKSSSTAHMELRSPTSEDTAVYYCARGYGNYVAYWGQGTLVTVSA

SEQ ID NO: 10 - protein, 117 aa, Artificial Sequence
  modified heavy chain variable region of murine antibody
EVQLQQSGPETGKPGASGKMSCKTSGHTSTEHNGQWAKQSPGQSLEWNGGRNNSIVKSITVSASGTKATLTADKSSSTAHMELRSPTSEDTAVYYCSPSYTYYRPGWGQGTLVTVSA

SEQ ID NO: 11 - protein, 117 aa, Artificial Sequence
  modified heavy chain variable region of murine antibody
EVQLQQSGPETGKFGATISFSCNTGYKLFGSTSGQWARQSPGQSLEWNGGRNNSIVKSITVSASGTKATLTADKSSSTAHMELRSPTSEDTAVYYCSPSYTYYRPGWGQGTLVTVSA

SEQ ID NO: 12 - protein, 117 aa, Artificial Sequence
  modified heavy chain variable region of murine antibody
EVQLQQSGPTLVKPTQTLTLTCTLSGFSFGSTSMNRLRQSPGQSLEWNGGRNNSIVKSITVSASGTKATLTADKSSSTAHMELRSPTSEDTAVYYCSPSYTYYRPGWGQGTLVTVSA

SEQ ID NO: 13 - protein, 107 aa, Artificial Sequence
  modified light chain variable region of murine antibody
DIQTTQSQKSQSTSAGDRASTTCKASQNVSTNAAWYQQTPGQSPKSLIYAASSLQSGVPDRFTGSGSGTDFTQTTSNAQSEDSAEFFCQQYNRYPHTFGGGTKLELK

SEQ ID NO: 14 - protein, 107 aa, Artificial Sequence
  modified light chain variable region of murine antibody
DIQTTQSQKSQSTSAGDRASTTCTLLSVTRNDVAWYQQTPGQSPKSLIYAASSLQSGVPDRFTGSGSGTDFTQTTSNAQSEDSAEFFCYLSGANLNLFGGGTKLELK

SEQ ID NO: 15 - protein, 17 aa, Homo sapiens
NITVNNSGSYTCQAHNS

SEQ ID NO: 16 - protein, 17 aa, Homo sapiens
NITVNNSGSYMCQAHNS

SEQ ID NO: 17 - protein, 17 aa, Homo sapiens
KITPNNNGTYACFVSNL

SEQ ID NO: 18 - protein, 17 aa, Homo sapiens
GRNNSIVKSITVSASGT

SEQ ID NO: 19 - protein, 8 aa, Hom sapiens
GYSWYKGE

SEQ ID NO: 20 - protein, 8 aa, Homo sapiens
SYTYYRPG

SEQ ID NO: 21 - protein, 8 aa, Homo sapiens
SKANYRPG

SEQ ID NO: 22 - protein, 8 aa, Hom sapiens
EDKDAVAF

SEQ ID NO: 23 - protein, 9 aa, Mus musculus
NVGSIYNQK

SEQ ID NO: 24 - protein, 9 aa, Homo sapines
IVKSITVSA

SEQ ID NO: 25 - protein, 9 aa, Mus musculus
INPNNVGSI

SEQ ID NO: 26 - protein, 10 aa, Homo sapiens
SIVKSITVSA

SEQ ID NO: 27 - protein, 9 aa, Mus musculus
VGSIYNQKF

SEQ ID NO: 28 - protein, 9 aa, Homo sapiens
SIVKSITVS

SEQ ID NO: 29 - protein, 8 aa, Homo sapiens
LATRNNSI

SEQ ID NO: 30 - protein, 6 aa, Mus musculus
VGSIYN

SEQ ID NO: 31 - protein, 7 aa, Homo sapiens
IVKSITV

SEQ ID NO: 32 - protein, 9 aa, Mus musculus
CARGYGNYV

SEQ ID NO: 33 - protein, 9 aa, Homo sapiens
HLFGYSWYK

SEQ ID NO: 34 - protein, 9 aa, Homo sapiens
NRFGYSWYK

(19) United States
(12) Patent Application Publication
(10) Pub. No.: US 2005/0222392 A1
(43) Pub. Date: Oct. 6, 2005

(54) Anti-idiotype anti-cea antibody molecules and its use as cancer vaccine

(76) Inventors: Graham Carter, Aberdeenshire (GB); Francis J. Carr, Aberdeenshire (GB)

Correspondence Address: OLSON & HIERL, LTD., 20 NORTH WACKER DRIVE, 36TH FLOOR, CHICAGO, IL, 60606, US

(21) Appl. No.: 10/510,523

(22) PCT Filed: Apr. 7, 2003
(86) PCT No.: PCT/EP03/03580

§ 371 Date: Apr. 28, 2005

(30) Foreign Application Priority Data
Apr. 9, 2002 (EP) 02007885.3

(51) Int. Cl. C07K 16/44

(52) U.S. Cl. 530/387.3; 530/388.8

(57) ABSTRACT

The present invention provides molecules, preferably designed immunoglubulins, suitable for use as an anti-idiotype vaccine to CEA positive tumours. The molecules induce both an MHC class I and MHC class II mediated immune response to the CEA bearing tumour cells for an efficient and sustained host anti-tumour response. The present invention provides modified versions of anti-idiotype anti-CEA antibodies, preferably mouse antibody 708, with improved vaccination properties. The modifications are related to the introduction of sequences tracts deriving from e.g. CEA, CD55 antigen and CEA cancer-specific MHC epitopes into the variable regions of said antibody molecules.

### FIELD OF THE INVENTION

The present invention provides molecules, preferably designed immunoglubulins, suitable for use as an anti-idiotype vaccine to CEA (carcinoembryonic antigen) positive tumours. The molecules induce both an MHC class I and MHC class II mediated immune response to the CEA bearing tumour cells for an efficient and sustained host anti-tumour response. The present invention provides modified versions of anti-idiotype anti-CEA antibodies, preferably mouse antibody 708, with improved vaccination properties. The modifications are related to the introduction of sequence tracts deriving from e.g. CEA, CD55 antigen and CEA cancer-specific MHC epitopes into the variable regions of said antibody molecules.

### BACKGROUND

There has been a long held desire to provide for compositions able to stimulate or amplify the interaction of the human immune system with cancer cells for the purpose of eliminating the cancer cells from the body. In contrast with vaccination to provide immunity to infectious agents, harnessing the immune system for the elimination of cancer cells is a more challenging technical objective, not least as the immune system is required to be directed to cells for which there is an established immunological tolerance or in some cases, the cancerous cells themselves may have gained properties rendering them able to evade normal immunological detection or elimination.

The present invention is concerned with the induction of T-cell dependent immune response to a cancer cell. Most previous work has focussed on CD8 positive T-cells and MHC class I restricted antigens, however the present invention recognises the importance of MHC class II restricted CD4 positive T-cell responses and in the preferred embodiment provides for a vaccine able to deliver both class I and class II restricted cancer antigen epitopes.

The cancer antigen targeted by molecules of the present invention is the carcinoembryonic antigen (CEA). CEA is a cell surface protein over-expressed by wide range of solid cancers and it has been the focus as a target for vaccine development by a number of different groups worldwide. The molecule is a gpi-anchored 180 kDa glycoprotein expressed by 90% of colorectal, 70% of gastric, pancreatic and non-small cell lung cancers and 50of breast cancers. The protein shows considerably homology with non-specific cross-reacting antigen (NCP) and the billiary glycoprotein (BGP) found on normal granulocytes. CEA can be detected in the circulation of a majority of patients with CEA positive tumours and it is also found in the normal digestive tract of the human foetus. The protein appears to function as an adhesion molecule and there is some expectation that therapies directed to CEA may be beneficial in preventing tumour metastasis. CEA is an attractive target for cancer immunotherapies, including vaccination schemes, as where it occurs it is typically present at high levels on the tumour surface.

A number of previous studies have exploited CEA derived protein sequences in a vaccination approach to therapy. Studies in mice have demonstrated the superiority of CEA expressed in vaccinia (rV-CEA) over recombinant CEA as a vaccine, and have shown induction cytotoxic T-lymphocyte (CTL) responses resulting in regression of established tumours [Kantor, J. et al (1992), J. Natl. Cancer lnst. 84: 1084-1091]. When applied to a phase I clinical study in patients with metastatic carcinoma, the rV-CEA was able to induce a CTL response to CEA that killed tumour cells [Tsang, K. Y. et al (1995) J. Natl. Cancer. Inst. 87: 982-990]. However a significant immune response to the vaccinia was also induced which limited the prospects for subsequent immunisations in these subjects to achieve a useful clinical outcome. Other clinical studies involving a priming dose with rV-CEA and then boosting with CEA encoded within an avipox vector has achieved promising responses in patients also receiving GM-CSF and low dose IL-2 [Marshall, J. L. et al (2000) J. Clin. Oncology. 18: 3964-3973]. Further clinical studies are required before the utility of such a complex vaccination regime can be demonstrated.

An alternative approach used to target CEA is anti-idiotype immunisation. Anti-idiotypic antibodies that recognise the binding site of anti-tumour antibodies can act as functional mimics of the antigen. They can therefore be used to stimulate both humoral and cellular responses. A phase I clinical trial of the murine anti-idiotype, 3H1 which mimics CEA, has been conducted in patients with advanced colorectal cancer. The 3H1 antibody has been described extensively in U.S. Pat. No. 5,977,315 and the antibody has been shown to induce anti-CEA antibody responses in patients, with a number showing proliferative responses to CEA [Foon, K. A. et al (1995) J. Clin. Invest. 96: 334-342]. Other studies treating patients with minimal residual disease, showed patients with T cell responses to both the anti-idiotypic antibody and CEA. In this study however, the anti-idiotype failed to elicit CTL responses [Foon, K. A., et al (1999) J. Clin. Oncology. 17: 2889-2895].

Anti-idiotype antibodies mimicking other tumour antigens than CEA have been clinically investigated for their utility as therapeutic vaccines. Examples include the GD2 antigen and the anti-idiotype antibody 1A7 [U.S. Pat No. 6,509,016], also anti-idiotype antibodies for the GD3 antigen [U.S. Pat. No. 5,529,922; EP0473721] and the melanoma associated p97 antigen [U.S. Pat. No. 4,918,164] to name but just a few. More complex adoptive immunotherapeutic methods exploiting anti-idiotypic antibodies have also been advanced for example as taught in U.S. Pat. No. 5,766,588.

One particular example of a vaccination scheme using an anti-idiotype antibody is provided by studies of the human monoclonal antibody 107AD5. This antibody has been found to provide a molecular mimic of the CD55 protein also known as tumour associated antigen 791T/gp72 found on colorectal cancer cells. The CD55 protein functions to protect cells from complement-mediated attack and in cancer cells this protein is commonly found at elevated levels [Li, L., et al (2001) Br. J. Cancer 84: 80-86]. The 107AD5 antibody has shown promise in a number of clinical trials and anti-tumour immune responses including IL-2 induction could be measured in a number of patients [Robins, R. A et al (1991) Cancer Res. 51: 5425-5429; Denton, G. W. L., et al (1994) Int J. Cancer 57: 10-14; & WO90104415]. More recent trials however have indicated that the antibody alone is not likely to be effective in patients with a large tumour burden and the vaccination strategy with this antibody may be more beneficial in patients carrying minimal residual disease [Maxwell-Armstrong, C. A. et al (2001) Bri. J. Cancer 84: 1443-1446].

Despite the evident progress, there remains a continued need for improved molecules able to elicit an immune response to human cancer cells in general and to CEA positive cancer cells and/or cancers positive for CD55 over-expression in particular.

### SUMMARY OF THE INVENTION

The present invention provides polypeptides suitable for use as an anti-idotype vaccine to CEA positive tumours. The inventors have recognised the importance of the need to induce both an MHC class I and MHC class if mediated immune response to the CEA bearing tumour cells for an efficient and sustained host anti-tumour response. The polypeptide compositions herein are able to provide both MHC class I and MHC class II restricted CEA epitopes.

The invention provides modified polypeptides wherein the polypeptide sequences are derived in large part from the murine anti-idiotype antibody 708. Where the polypeptide sequences share sequence tracts in common with the V-regions of antibody 708 there are provided a number of embodiments in which sequence tracts from either CEA and/or the CD55 antigen are additionally provided. In a further embodiment there are provided polypeptide sequences in which amino acid substitutions have been conducted to result in the removal of undesired T-cell epitopes. In such compositions the intent is to focus the induced immune response to the CEA and/or CD55 epitope component and remove competing peptide epitopes not contributing to the desired anti-cancer response.

The parental 708 antibody was produced using anti-CEA antibody NCRC23 as antigen. The NCRC23 monoclonal antibody itself binds to a CEA specific epitope and shows minimal cross-reactivity with normal tissues [Price, M. R. et al (1987), Cancer, Immunology and Immunotherapy. 25: 10-15]. Anti-idiotypic antibody 708 specifically recognises NCRC23 and can induce Ab3 antibodies in mice and rats that recognise CEA. Of particular significance is that the 708 anti-idiotype antibody can also prime human T lymphocytes from cancer patients to recognise either CEA or CEA expressing tumour cells [Durrant, L. G. et al (1992), Int. J. Cancer. 50: 811-816].

The variable region sequences of the 708 antibody have been obtained and analysed for the presence of sequence elements homologous to regions of the CEA protein. The first and second complementarity determining regions of the H-chain (CDRH2 and CDRH3) show homology with CEA but not to the closely related molecules NCA or BGP. The 708 variable region and the complementarity determining regions (CDRs) of the H-chain in particular represent a molecular mimic of particular elements of the CEA molecule and are likely to provide the basis for the idotypic nature of the 708 antibody for CEA.

The present invention comprises modified derivative versions of the parental antibody 708. In all preferred embodiments the modified 708 molecules include a human C-region domain in place of the parental murine C-regions. Other modifications are conducted in the V-region domains of the molecule. Such modifications can be summarised as comprising one or more changes directed towards the following objectives, wherein at least on change directed to a CEA sequence has to be involved:

I. Conversion of regions of existing CEA homology into regions CEA sequence identity.

II. Replacement of existing short sequence tracts with tracts of CEA derived sequence.

III. Replacement of existing short sequence tracts with tracts of antibody 107AD5 derived sequence.

IV. Replacement of existing short sequence tracts with tracts of CD55 derived sequence.

V. Removal of undesired T-cell epitopes by replacement of specific amino acid residues with alternative amino acid residues.

The present invention thereby provides new polypeptide sequences each designed according to one or more of the above objectives and each featuring sequences elements with identity or close homology to the native 708 V-regions, the human CEA molecule, and/or the human CD55 molecule or an idiotype to the CD55 molecule in the form of mouse antibody 107AD5. The invention incorporates a number of polypeptide sequences which together encompass all of the above listed “design elements”. Each polypeptide disclosed herein is an embodiment according to the invention.

In summary the invention is concerned with the following issues:
An immunoglobulin molecule or a fragment thereof deriving from a parental anti-idiotype anti-CEA antibody and comprising constant regions from human origin and synthetically designed variable regions comprising one or more sequence tracts of more than 4, preferably 5-20, consecutive amino acid residues deriving from human tumour antigen CEA (carcinoembryonic antigen). Most preferred are sequence tracts having exactly the length (number of the amino acid residues) of a CDR of a light or heavy chain of the corresponding anti-idiotype antibody (e.g. 5, 7, 8, 9, 10, 11, 12, 17, 18)
A corresponding immunoglobulin molecule, wherein at least one of said sequence tracts is a component of a complementarity determining region (CDR) of the heavy and/or light chain of said immunoglobulin or overlaps with adjacent residues of a framework region adjacent to said CDR.
A corresponding immunoglobulin molecule, wherein said component forms 30 to 100%, preferably 80-100%, of the amino acid residues of said CDR.
A corresponding immunoglobulin molecule, wherein said parental anti-idiotype antibody is mouse antibody 708. However, also other anti-idiotype anti-CEA antibodies are suitable according to the invention.
A corresponding immunoglobulin molecule, comprising within the variable regions additionally sequence tracts of 5 to 25, preferably 10 to 20, consecutive amino acid residues deriving from human CD55 antigen or the hypervariable regions of an anti-idotype anti-CD55 antibody, wherein antibody 105AD7 is preferred.
A corresponding immunoglobulin molecule, wherein within the variable regions additionally potential MHC class II epitopes, which do not contribute to an immune response to CEA positive human cancer cells, have been removed by amino acid substitutions.
A corresponding immumoglobpulin molecule comprising within the variable regions additionally CEA derived sequence tracts which are MHC class I epitopes responding to CEA positive human cancer cells, preferably TLLSVTRNDV and YLSGANLNL, where in a preferred embodiment of the invention said sequences are part of or form completely one ore more of the CDRs of the light chain of said immunoglobulin.
A corresponding immunoglobulin molecule comprising within the variable regions additionally CEA derived sequence tracts which are MHC class II epitopes which contribute to an immune response to CEA positive human cancer cells.
A corresponding immunoglobulin molecule comprising a variable heavy chain selected from any of the sequences as depicted in FIGS 4 to 7 and/or a variable light chain selected from any of the sequences as depicted in FIGS. 8 and 9.
A corresponding immunoglobulin molecule, wherein the variable heavy and/or light chain comprises one or more sequence tracts in identity with the sequence tracts selected from the group:
   (i) 345-354 of human CEA;
   (ii) 387-396 of human CEA
   (iii) 571-579 of human CEA
   (iv) 629-645 of human CEA
   (v) 148-167 of human CD55.
A pharmaceutical composition comprising an immunoglobulin molecule as described above in an biologically effective amount, an adjuvant, and optionally a pharmaceutically acceptable carrier, diluent or excipient.
The use of an immunoglobulin molecule or a pharmaceutical composition of any of the above-specified claims for the manufacture of a medicament for vaccination of a human individual suffering from a CEA positive solid or metastasising tumour, wherein preferably said vaccination causes improved stimulation of CD8 and/or CD4 positive T-cells in said individual.
A method for the production of a vaccine molecule based on a synthetically designed immunoglobulin molecule suitable for the treatment of a human individual suffering from a CEA (carcinoembryonic antigen) positive solid or metastasising tumour, said method comprising the following steps:
   (i) selecting a non-human anti-idiotype anti-CEA antibody, preferably mouse antibody 708,
   (ii) replacing the non-human constant regions by a human constant regions,
   (iii) replacing partially or completely one or more of the hypervariable regions (CDRs), with sequence tracts deriving from CEA, whereby framework residues adjacent to said CDRs may be included, and optionally comprising one or more of the steps selected from the group:
   (iv) replacing sequence tracts within the variable regions with tracts deriving from CD55 antigen or the CDRs of an anti-idiotype anti-CD55 antibody.
   (v) replacing sequence tracts within the variable regions with tracts which are MHC class I and/or MHC class II epitopes contributing to an immune response directed to CEA positivehuman cancer cells,
   (vi) removing within the variable regions potential MHC class II epitopes, which do not contribute to an immune response to CEA positive human cancer cells.

A first embodiment of the invention is provision of an antibody molecule comprising antibody 708 with human constant regions.

A second embodiment is provision of antibody 708 with human constant regions and featuring modification of the V-region domains. The preferred modifications are conducted within one or more of the CDR regions of the molecule and result in the presence of sequence tracts with identity to human CEA. Such CEA sequence elements are considered “desired” epitopes. In further embodiments the number of desired CEA epitopes is increased further by introduction of other CEA derived sequence elements. In yet further embodiments alternative desired epitopes are additionally included into the sequence by substitution of amino acid residues. Particularly desired alternative epitopes are sequence elements from the human CD55 antigen or the mouse antibody termed 105AD7 which itself is an anti-idiotypic monoclonal antibody that itself provides a molecular mimic of the CD55 protein [Maxwell-Armstrong, C. A., et al (2001) Bri. J. Cancer 84: 1433-1436]. Such desired additional epitopes are inserted into the antibody V-region at positions which may include CDRs and or adjacent framework domains.

In a further embodiment of the invention there are provided antibody sequences comprising one or more desired epitope sequences within a V-region domain depleted of undesired epitope sequences. Such sequences in this instance are MHC class II directed epitopes and are removed by judicial amino acid substitutions within the peptide constituting a ligand for at least one MHC class II allotype extant in the human population.

Under the scheme of the present there are provided 4 different H-chain V-region sequences and 2 different L-chain V-region sequences. The present disclosure provides no limit to the possible combinations of H-chain and L-chain that may be provided to constitute a complete antibody molecule. Constitution of the complete antibody molecule may be achieved by recombinant DNA techniques and methods for purifying and manipulating antibody molecules well known in the art. Polynucleotide (e.g. DNA) molecules encoding the polypeptide sequences disclosed herein are equally considered under the scope of the present and are preferred embodiments.

The antibody molecules of the present invention are intended for use intact but this is not meant to be a limitation and immunogenic fragments of the antibodies may also be considered for use. Therefore Fv, Fab or F(ab′)2 or other derivatives may be prepared using recombinant techniques or fragments prepared using conventional techniques of antibody proteolytic cleavage and purification.

It is an objective of the invention that the antibodies disclosed herein find utility in compositions containing an immunogenic and most preferably a therapeutic amount of at least one of the modified antibody molecules of the invention. The immunogenic or therapeutic amount is a quantity of the antibody composition able to stimulate an immune response in a patient receiving the therapy and in whom the immune response is most preferably both a humoral and a cellular response. It is most desired to provide a composition in which the therapeutic amount results in the patients immune system exhibiting increased activity against tumour cells expressing CEA. The compositions will have a therapeutic effect in eliminating tumour cells or arresting tumour growth.

### BRIEF DESCRIPTION OF THE FIGURES

FIG. 1 provides a sequence comparison of the CDR regions of 708 anti-idiotypic antibody and CEA. The bold amino acids are those which show identify and the underlined those of identify or similarity in the next amino acid.

FIG. 2 provides examples of MHC binding motif analysis of the CDR2 and CDR3 variable regions of the heavy chain of the 708 anti-idiotype. The bold amino acids are those which show identity and the underlined those of identity or similarity in the next amino acid.

FIG. 3 shows the protein sequence (single letter code) of the variable regions of antibody 708. A=heavy chain; B=light chain. Underlined sequences are CDRs. FR=framework sequence. CDR designations are according to the scheme of Kabat [Martin, A. C. R. (1996), PROTEINS: Structure, Function and Genetics, 25 130-133] but residue numbering has been modified individually according to this invention.

FIG. 4 shows the protein sequence (single letter code) of 708VH1. This sequence comprises 708VH, with un-desired epitopes removed. Underlined sequences are CDRs.

FIG. 5 shows the protein sequence (single letter code) of 708VH2. This sequence comprises 708VH, with un-desired epitopes removed and incorporating additional CEA related sequences. Underlined sequences are CDRs.

FIG. 6 shows the protein sequence (single letter code) of 708VH3. This sequence comprises 708VH, with undesired epitopes removed and incorporating additional CEA and CD55 derived sequences. Underlined sequences are CDRs.

FIG. 7 shows the protein sequence (single letter code) of 708VH4. This sequence comprises 708VH, with undesired epitopes removed and incorporating additional CEA and 105AD7 derived sequences. Underlined sequences are CDRs.

FIG. 8 shows the protein sequence (single letter code) of 708VL4. This sequence comprises 708VL, with un-desired epitopes removed. Underlined sequences are CDRs.

FIG. 9 shows the protein sequence (single letter code) of 708VL. This sequence comprises 708VL, with un-desired epitopes removed and incorporating additional CEA related sequences. Underlined sequences are CDRs.

FIG. 10 shows the protein sequence (single letter code) of CEA.

FIG. 11 shows protein sequence (single letter code) of CD55 antigen.

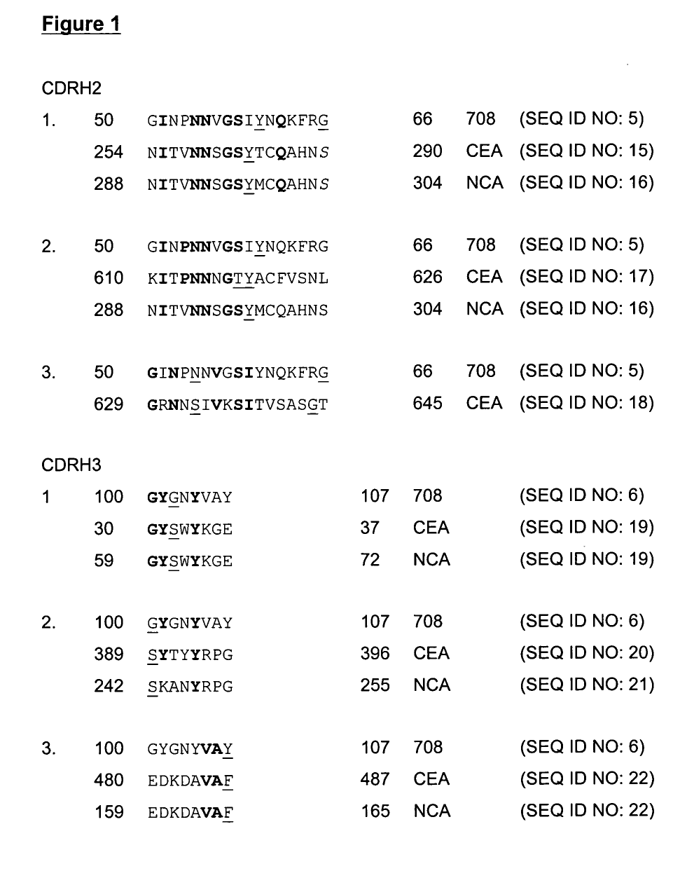

### DETAILED DESCRIPTION OF THE INVENTION

The molecules of the present invention are modified antibody molecules with utility as the active components of an anti-cancer vaccine. The invention is therefore concerned with the therapeutic treatment of human disease. The molecules originate as an anti-idiotypic antibody termed 708. The 708 monoclonal antibody was raised against an anti-CEA monoclonal antibody NCRC23. The native 708 antibody is able to block the interaction of NCRC23 with its antigen and can induce both antibody and T cell responses that specifically recognise this antigen, however the native mouse 708 antibody could not stimulate lymphocytes from normal donors [Durrant, L. G. et al (1992), ibid]. A number of modifications have been made to the native 708 antibody in order to improve its capability to function as an anti-cancer vaccine. The modifications have resulted in the compositions disclosed herein and are embodiments of the present invention. All modifications to the native (parental) mouse 708 antibody may be conducted using genetic engineering means widely known in the art.

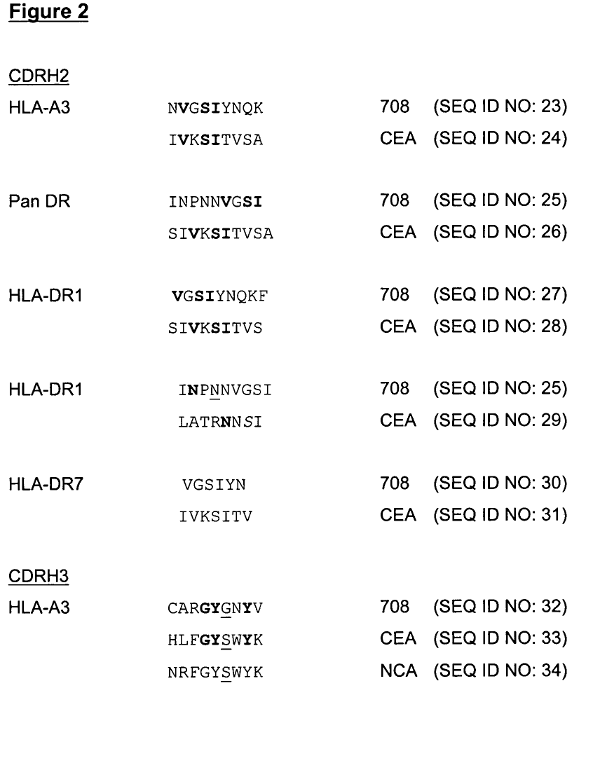

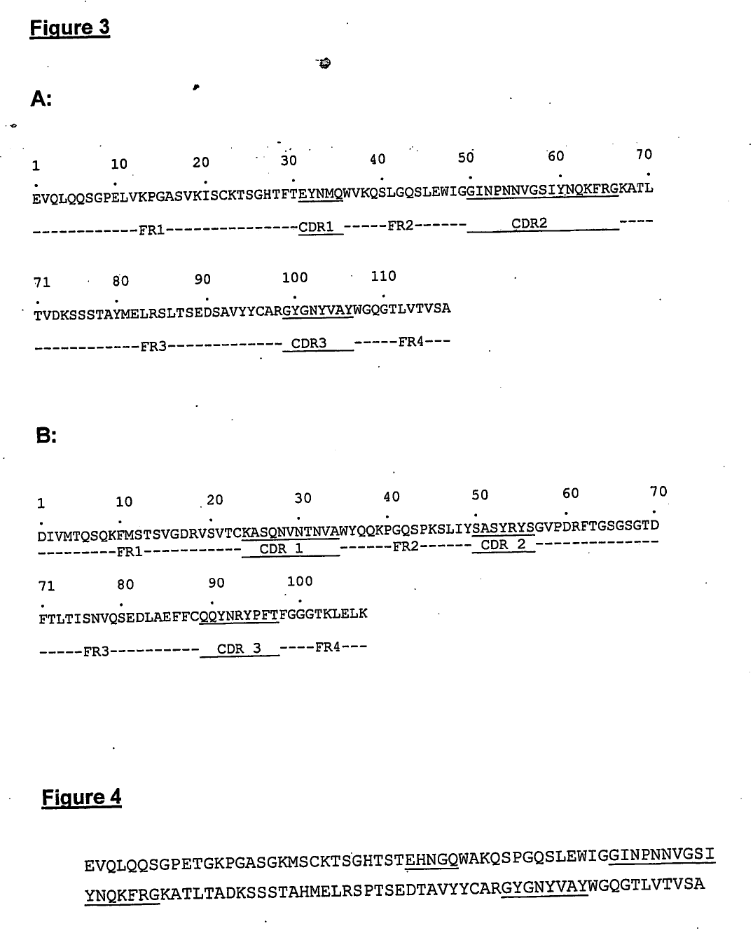

The first such modification is common to each of the variant 708 molecules. This modification is the engineering of the constant region domains such that these are human constant region protein sequences. It is common in the field to term such an engineered antibody a chimeric antibody. Within the context of the present invention, conversion of the murine 708 antibody to a chimeric antibody has a very significant consequence with respect to the ability of the modified 708 molecule to act as an anti-cancer vaccine. The inventors have recognised that stimulation of naïve T cell responses requires good targeting of antigen presenting cells such as dendritic cells. The human constant region domain of each of the modified 708 molecules of the present invention enables uptake of the molecules via the Fc (CD64) receptors on dendtritic and other cells. Uptake via this route has been shown to result in priming of both helper and cytotoxic T cell responses [Durrant, L. G., et al (2001), Int. J. Cancer. 92: 414-420]. In the present case a human IgG1 isotype has been engineered to 708 derived V-regions although in principle it is understood that any isotype able to be recognised by the Fc receptor system could be incorporated under the scheme of the present invention.

Where the first modification of the 708 antibody is conversion to a chimaeric antibody and therefore involved engineering of the constant region, subsequent modifications, and hence embodiments of the invention, are directed towards engineering of the V-regions of the parental 708 antibody. The V-region sequences of 708 have been described previously (WO981529761 and the protein sequences are again provided herein as FIG. 3. The complementarity determining region (CDR) sequences have been analysed for regions of homology with CEA and related sequences such as NCA. The CDRH2 shows homology with three specific regions of CEA and two of these also share homology with NCA. A third region is in an area specific to CEA. As the original Ab1 NCRC23, bound to a CEA specific region it is not unexpected to find that the anti-idiotype 708 should contain CEA-homlogous sequence. In addition to the region found in CDRH2, the CDRH3 showed homology with three regions of CEA, and these also share homology with NCA. Comparative analysis of polypeptide and polynucleotide sequences is well known in the art and a number of software tools enable these procedures. One such, as used for the comparison of the antibody 708 and. CEA sequences as described above, is “DNAstar”, (DNASTAR Inc, Madison, Wis., USA) which has implementations of several alignment algorithms including Lipman & Pearson [Lipman & Pearson (1985), Science 227:1435-1441] which is particularly useful for protein sequence similarities.

Analysis has also been made for regions of the antibody 708 heavy and light chains and human CEA that conform toe recognised T-cell epitope motifs. Such analysis may be conducted using methods known in the art for example as described in WO98/52976 or reference to databases such as SYFPEITHI [Rammensee, H. G. et al (1999) Immunogenetics 50: 213-219]. One analysis shows the CDRH2 region containing HLA-A3, A11, Aw68, B35, B53, DR1, DR3, DR7 and DR8 binding motifs. Analysis of CEA sequence in parallel confirmed the HLA-A3, DR1 and DR7 motifs are also present in the CEA specific area with homology to CDRH2. The CDRH3 region contained HLA-A2, A3, A11, A24, B27, DQ7, pan DR and DR 1 binding motifs. The HLA-A3 motif was also found in the homologous region of CEA. Although this region of CEA also shows homology with NCA there is an amino acid difference in NCA from the leucine to an arginine. As the leucine is a key pocket residue for A3 binding it is unlikely that cells expressing NCA will present this eptiope in the context of HLA-A3. These results suggested that patients with HLA-DR1 or 7 and HLA-A3 phenotypes should show both helper and cytotoxic T cell responses to the native 708 and are most likely to respond to their CEA positive tumours.

Taken together, these results and observations have lead to an understanding that the native 708 V-region sequences provide a molecular mimic of the CEA molecule. Furthermore the mimicry appears to be directed to a number of different locations on the CEA sequence and in turn these sequences conform to a number T-helper and cytotoxic T-cell type motifs. The inventors have sought to increase the inherent CEA-like immunogenic profile of the native 708 sequence by making sequence modifications so as to increase the degree of CEA like sequence within the molecule. The strategy has been extended to include seeding additional CEA derived sequence elements into the parental 708 V-region at positions where no pre-existing homology with the in-coming CEA derived sequence is present. This has been made possible by the inherent flexibility of the immunoglobulin V-region in being able to accept sequence hypervariability at particular zones (the CDRs) and yet retain overall structural integrity and the ability to be expressed and processed as any other immunoglobulin molecule. This process is further aided by the lack of any requirement for the engineered antibody to retain any form of antigen binding activity, indeed it is most preferred that no interaction with any antigen, especially a cell binding antigen, is possible by the preparations of this invention.

The polypeptide molecules of the present are designed with the purpose of providing immunogenic epitopes to the immune system of the subject patient such that the patients immune system becomes re-directed to eliminate cells expressing CEA. It is important therefore to evoke humoral and cellular arms of the immune system and this is provided by delivery of both potent T-helper epitopes and MHC class 1 restricted epitopes. A number of MHC class I restricted epitopes have been identified previously within the CEA sequence and in some instances have been the subject of clinical trial [Kwong, Y. et al (1995) JNCI 87: 982-990]. In the present invention, CEA derived sequence tracts TLLSVTRNDV (residues 345-353) and YLSGANLNL (residues 571-579) which are known MHC class I epitopes have been engineered into the CDRs of the light chain.

In addition to the use of CEA derived sequence tracts, a further important feature of the present invention is incorporation of CD55 sequences and a mimetic version of part of CD55 in the form of sequence tracts from antibody 105AD7. The CD55 molecule is widely expressed in normal human tissues where it serves to protect cells from complement and natural killer (NK) cell mediated lysis. The validity of CD55 as a target for immunotherapy stems from the observation of increased expression of CD55 on multiple tumour types and studies using antibody 105AD7. This antibody mimics an epitope on CD55 and clinical trails have demonstrated stimulation of T-cell responses in patients treated with the whole 105AD7 antibody in a vaccination strategy [WO97/32021 and all references therein].

The present invention for the first time provides compositions featuring combinations of immunogenic epitopes derived from CEA, CD55 and/or 105AD7. Moreover the epitopes each with proven biological potency with respect to stimulation of human immune responses are provided as part of an immunoglobulin molecule to confer significant technical and biological advantages over schemes for example where the equivalent epitopes are provided individually as synthetic peptides. As molecular entities the polypeptides of the present invention could be described as “antigenised antibodies”. In the literature there are reported antigenised antibodies included antibodies featuring combination of MHC class I and MHC class II type epitopes [Zaghouani, H. et al (1993) Eur. J. Immunol. 23: 2746-2750; Xiong, S. et al (1997) Nature Biotech, 15: 882-886 and references therein]. It is the inventors understanding that none of these approaches have been directed to self antigens, exploited idotypic determinants, nor taken steps to deplete the non engineered sequence of un-desired immunogenic epitopes according to the scheme of the present. Equally, none of these previous studies have provided compositions directed to cancer immunotherapy.

Accordingly the invention provides modified V-region sequences containing tracts of sequence which share identity to regions of the CEA molecule. The invention also discloses V-region sequences that share identity with tracts of sequence present in the CD55 molecule. In a further embodiment still, there is disclosed a V-region sequence modified to contain a sequence tract from the antibody 107AD5. Specifically there are provided V-region sequences containing residues in identity with residues 345-354, 386-397, 571-579 and 629-645 from the CEA sequence; and sequences in identity with residues 148-167 of the CD55 molecule. A sequence corresponding to the majority of framework 1 of the VH chain of antibody 107AD5 is incorporated within one disclosed variant of the present. Specifically a composition according to the sequence of FIG. 7 is preferred and contains sequence elements of the 107AD5 VH framework 1 region in replacement of the corresponding region within the 708VH3 sequence described herein.

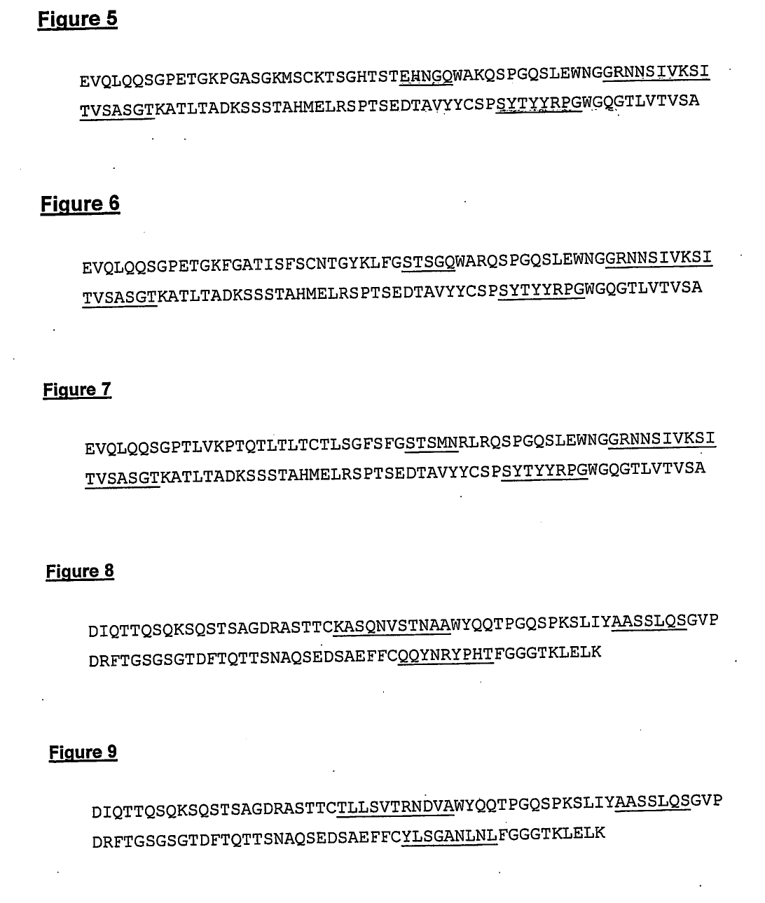

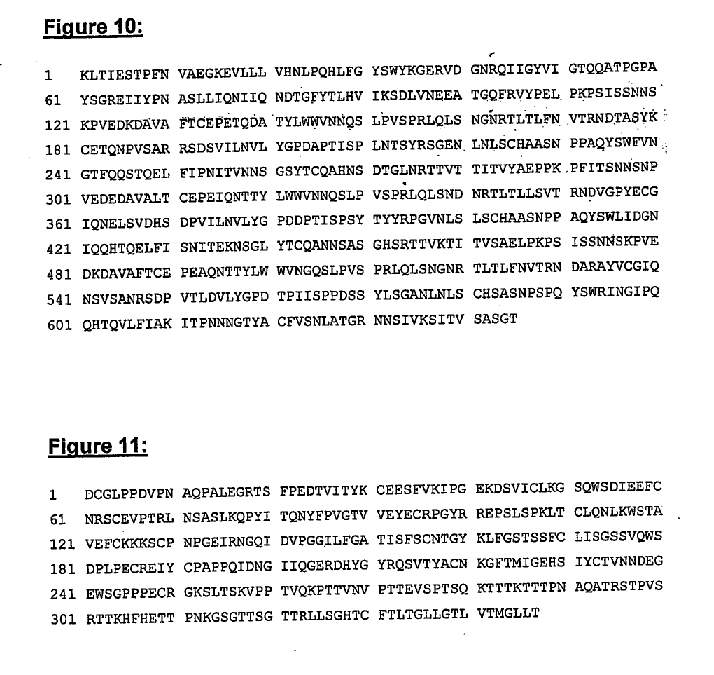

It will be appreciated that for the CEA sequence elements inserted into the VH chains of the modified 708 molecule, the insertions have been into regions where significant homology to the CEA sequence existed in the parent molecule. Thus a preferred VH composition as shown in FIG. 5 comprises CEA residues 629-645 inserted into the VH chain at a zone encompassing the CDRH2 region, and also includes CEA residues 386-397 inserted into the VH chain at a zone encompassing the CDRH3 region. A preferred VL chain composition provides CEA sequence elements 345-354 and 571-579 inserted into the VL chain at regions encompassing the CDRL1 and CDRL3 zones respectively (FIG. 9). A preferred composition containing CD55 sequence elements such as region 148-167 contains the said CD55 sequence inserted into a VH chain within a zone comprising the distal part of framework 1 and the entirety of CDRH1 (FIG. 6).

It is understood that herein, the term “immunogencity” includes an ability to provoke, induce or otherwise facilitate a humoral and or T-cell mediated response in a host animal and in particular where the “host animal” is a human.

The term “antibody” or “immunoglobulin” herein is used in the broadest sense and specifically covers intact monoclonal antibodies, polyclonal antibodies, multispecific antibodies (e.g. bispecific antibodies) formed from at least two intact antibodies, and antibody fragments, so long as they exhibit the desired biological activity. The term generally includes heteroantibodies which are composed of two or more antibodies or fragments thereof of different binding specificity which are linked together.

Depending on the amino acid sequence of their constant regions, intact antibodies can be assigned to different “antibody (immunoglobulin) classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ and μ respectively. Preferred major class for antibodies according to the invention is IgG, in more detail IgG1 and IgG2.

Antibodies are usually glycoproteins having a molecular weight of about 150,000, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (VH) followed by a number of constant domains. The variable regions comprise hypervariable regions or “CDR” regions, which contain the antigen binding site and are responsible for the specificity of the antibody, and the “FR” regions, which are important with respect to the affinity/avidity of the antibody. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). The “FR” residues (frame work region) are those variable domain residues other than the hypervariable region residues as herein defined. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains. The “light chains” of antibodies from any vertebrate species can be assigned to one of two clearly distinct types, called kappa (κ) and lambda (λ), based on the amino acid sequences of their constant domains.

The term “complementarity determining region” (CDR) refers to the segments of sequence within antibody V-regions which are hypervariable in sequence relative to the rest of the V-region domain. The CDRs of antigen binding antibodies are critical in determining the antibody antigen interaction. Each V-region contains three CDRs and by convention CDRs from the VH are termed CDRH1, CDRH2 and CDRH3. Similarly light chain CDRs are termed CDRL1, CDRL2 and CDRL3. The CDRs are interspersed by regions of relatively invariant sequence termed “framework” (FR) segments or domains.

In the present invention, modifications have been made both to the CDRs and framework regions of both VH and VL chains. Some of the modifications are dispersed single amino acid substitutions and in other cases, tracts of new sequence have been inserted into the parental V-region sequence.

As used herein, VH means a polypeptide that is about 110 to 125 amino acid residues in length, the sequence of which corresponds to any of the specified VH chains herein which in combination with a VL are capable of constituting an immunoglobulin molecule. Similarly, VL means a polypeptide that is about 95-130 amino acid residues in length the sequence of which corresponds to any of the specified VL chains herein which in combination with a VH are capable of co-association and constitution of the full immunoglobulin tetramer. Full-length immunoglobulin heavy chains are about 50 kDa molecular weight and are encoded by a VH gene at the N-terminus and one of the constant region genes (e.g. γ) at the C-terminus. Similarly, full-length light chains are about 25 kDa molecular weight and are encoded by a V-region gene at the N-terminus and a κor λ constant region gene at the C-terminus.

In the art the term “antibody” is accepted to indicate a molecule that is capable of combining, interacting or otherwise associating with an antigen, and the term “antigen” is used to refer to a substance that is capable of interacting with the antibody.

It will be readily recognised that within the context of the present invention, the modified immunoglobulin sequences as defined above and as follows are constructed to serve as vehicles for the delivery of specific immunogenic peptide sequences and there is no expectation or desire that an immunoglobulin arising from the combination of any of the polypeptide sequences disclosed herein could function as a binding entity for an antigen. In all respects other than antigen binding, the molecules disclosed herein retain the same domain structure and constant region sequences as antibodies and thereby continue to be considered as antibodies.

The term “monoclonal antibody” as used herein refers to an antibody obtained from a population of substantially homogeneous antibodies, ie., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. Methods for making monoclonal antibodies include the hybridoma method described by Kohler and Milstein (1975, Nature 256, 495) and in “Monoclonal Antibody Technology. The Production and Characterization of Rodent and Human Hybridomas” (1985, Burdon et al., Eds. Laboratory Techniques in Biochemistry and Molecular Biology, Volume 13, Elsevier Science Publishers, Amsterdam), or may be made by well known recombinant DNA methods (see, e.g., U.S. Pat. No. 4,816,567). Monoclonal antibodies may also be isolated from phage.antibody libraries using the techniques described in Clackson et al., Nature, 352:624-628 (1991) and Marks et al., J. Mol. Biol., 222:58, 1-597(1991), for example.

The term “chimeric antibody” means antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g.: U.S. Pat. No. 4,816,567; Morrison et al., Proc. Nat Acad. Sci. USA, 81:6851-6855 (1984)). Methods for making chimeric and humanized antibodies are also known in the art. For example, methods for making chimeric antibodies include those described in patents by Boss (Celltech) and by Cabilly (Genentech) (U.S. Pat. Nos. 4,816,397; 4,816,567).

The immunoglobulins od the present invention may be complete antibodies or fragments thereof. “Antibody fragments” comprise a portion of an intact antibody, preferably comprising the antigen-binding or variable region thereof. Examples of antibody fragments include Fab, Fab′, F(ab′)2, Fv and Fc fragments, diabodies, linear antibodies, single-chain antibody molecules; and multispecific antibodies formed from antibody fragment(s). An “intact” antibody is one which comprises an antigen-binding variable region as well as a light chain constant domain (CL) and heavy chain constant domains, CH1, CH2 and CH3. Preferably, the intact antibody has one or more effector functions. Papain digestion of antibodies produces two identical antigen-binding fragments, called “Fab” fragments, each comprising a single antigen-binding site and a CL and a CH1 region, and a residual “Fc” fragment, whose name reflects its ability to crystallize readily. The “Fc” region of the antibodies comprises, as a rule, a CH2, CH3 and the hinge region of an IgG1 or IgG2 antibody major class. The hinge region is a group of 15 amino acid residues which combine the CH1 region with the CH2-CH3 region. Pepsin treatment yields an “F(ab′)2” fragment that has two antigen-binding sites and is still capable of cross-linking antigen. “Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions (CDRs) of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. The Fab fragment also contains the constant domain of the light chain and the first constant domain (CH1) of the heavy chain. “Fab′” fragments differ from Fab fragments by the addition of a few residues at the carboxy terminus of the heavy chain CH1 domain including one or more cysteines from the antibody hinge region. F(ab′)2 antibody fragments originally were produced as pairs of Fab′ fragments which have hinge cysteines between them. Other chemical couplings of antibody fragments are also known (see e.g. Hermanson, Bioconjugate Techniques, Academic Press, 1996; . U.S. Pat. No. 4,342,566). “Single-chain Fv” or “scFv” antibody fragments comprise the V, and V, domains of antibody, wherein these domains are present in a Single polypeptide chain. Preferably, the Fv polypeptide further comprises a polypeptide linker between the VH and VL domains which enables the scFv to form the desired structure for antigen binding. Single-chain FV antibodies are known, for example, from Plückthun (The Pharmacology of Monoclonal Antibodies, Vol. 113, Rosenburg and Moore eds., Springer-Verlag, New York, pp. 269-315 (1994)), WO93/16185; U.S. Pat. Nos. 5,571,894; 5,587,458; Huston et al. (1988, Proc. Natl. Acad. Sci. 85, 5879) or Skerra and Plueckthun (1988, Science 240, 1038).

The immunoglobulins of the invention may be also bispecific antibodies. Bispecific antibodies” are single, divalent antibodies (or immunotherapeutically effective fragments thereof) which have two differently specific antigen binding sites. For example the first antigen binding site is directed to an angiogenesis receptor (e.g. integrin or VEGF receptor), whereas the second antigen binding site is directed to an ErbB receptor (e.g. EGFR or Her 2). Bispecific antibodies can be produced by chemical techniques (see e.g., Kranz et al. (1981) Proc. Natl. Acad. Sci. USA 78, 5807), by “polydoma” techniques (See U.S. Pat. No. 4,474,893) os by recombinant DNA techniques, which all are known per se. Further methods are described in WO 91/00360, WO 92/05793 and WO 96/04305. Bispecific antibodies can also be prepared from single chain antibodies (see e.g., Huston et al. (1988) Proc. Natl. Acad. Sci. 85, 5879; Skerra and Plueckthun (1988) Science 240, 1038).

The immunoglobulins of the invention may be also immunoconjugates. The term “immunoconjugate” refers to an antibody or immunoglobulin respectively, or a immunologically effective fragment thereof, which is fused by covalent linkage to a non-immunologically effective molecule. Preferably this fusion partner is a peptide or a protein, which may be glycosylated. Said non-antibody molecule can be linked to the C-terminal of the constant heavy chains of the antibody or to the N-terminals of the variable light and/or heavy chains. The fusion partners can be linked via a linker molecule, which is, as a rule, a 3-15 amino acid residues containing peptide. Immunoconjugates according to the invention consist of an immunoglobulin or immunotherapeutically effective fragment thereof, directed to a receptor tyrosine kinase, preferably an ErbB (ErbB1/ErbB2) receptor and an integrin antagonistic peptide, or an angiogenic receptor, preferably an integrin or VEGF receptor and TNFα or a fusion protein consisting essentially of TNFα and IFNγ or another suitable cytokine, which is linked with its N-terminal to the C-terminal of said immunoglobulin, preferably the Fc portion thereof. The term includes also corresponding fusion constructs comprising bi- or multi-specific immunoglobulins (antibodies) or fragments thereof.

The antibody molecules of the present invention are conceived to function as the active (i.e. immunogenic) component of a vaccine preparation, wherein the term “vaccine” describes a preparation for administration to a subject for the purpose of inducing an immune reaction. In the present context the immune reaction is with therapeutic intent although vaccines may be used as an adjunctive therapy to surgical removal of a tumour or for the prophylaxis of disease or relapsing disease.

The term “T-cell epitope” means according to the understanding of this invention an amino acid sequence which is able to bind MHC class I or class II, able to stimulate T-cells and or also to bind (without necessarily measurably activating) T-cells in complex with MHC class I or class II.

The term “peptide” as used herein and in the appended claims, is a compound that includes two or more amino acids. The amino acids are linked together by a peptide bond (defined herein below). There are 20 different naturally occurring amino acids involved in the biological production of peptides, and any number of them may be linked in any order to form a peptide chain or ring. The naturally occurring amino acids employed in the biological production of peptides all have the L-configuration. Synthetic peptides can be prepared employing conventional synthetic methods, utilizing L-amino acids, D-amino acids, or various combinations of amino acids of the two different configurations. Some peptides contain only a few amino acid units. Short peptides, e.g., having less than ten amino acid units, are sometimes referred to as “oligopeptides”. Other peptides contain a large number of amino acid residues, e.g. up to 100 or more, and are referred to as “polypeptides”. By convention, a “polypeptide” may be considered as any peptide chain containing three or more amino acids, whereas a “oligopeptide” is usually considered as a particular type of “short” polypeptide. Thus, as used herein, it is understood that any reference to a “polypeptide” also includes an oligopeptide. Further, any reference to a “peptide” includes polypeptides, oligopeptides, and proteins. Each different arrangement of amino acids forms different polypeptides or proteins. The number of polypeptides-and hence the number of different proteins that can be formed is practically unlimited.

The present invention provides for a series of modified VH and modified VL sequences. As previously stated, an antibody molecule of the IgG type comprises two H-chains and two L-chains in association by disuphide linkage. It will be appareciated that in principle any combination of H-chain and L-chain can be made and one route would be the co-expression of the relevant antibody genes from within the same cell. For the various H-chain and L-chain sequences disclosed in the present invention there is not intended to be a limit on the combination of any particular H-chain with any particular L-chain although one particularly preferred set of combinations would be that of H-chain 1 with L-chain 1; H-chain 2 with L-chain 2, H-chain 3 with L-chain 2 and H-chain 4 with L-chain 2. Other combinations may be contemplated and could for example include combinations featuring either of the parental 708 V-regions of FIG. 3.

For specific delivery of protein-derived antigens to MHC class I or class II molecules, the protein must be processed correctly within an appropriate compartment for subsequent release and presentation of peptides on MHC class I and class II molecules. The presence of human constant region domains and particularly the preferred IgG1 isotype of the molecules of the present invention maximise the opportunity for the protein to enter the antigen presenting cell (APC) where it will be taken up via the Fc (CD65) surface receptor. In general, peptide presentation of MHC class I is facilitated if the protein is processed in the cytoplasm whilst presentation on MHC class II is facilitated if the protein is processed in the endosomal compartments. Exogenous protein antigens often give rise to a good MHC class II-mediated responses (especially helper T cell expansion) but poor MHC class I-mediated responses. Uptake via the Fc (CD65) receptor represents a special case and results in optimal presentation of both class I and class II epitopes [Durrant, L. G. (2001), ibid].

A common feature of both MHC class I and MHC class II restricted tissue-specific peptides such as arising from the CEA protein and may be recognised by T-cells, is their low affinity for the MHC peptide binding groove [Pardoll, D. (1998) Nat. Medicine 4: 525-531]. Such epitopes are therefore, in relative terms, presented with low efficiency to the surface of the APC and their cognate T-cell population may have not been rendered tolerant to the self-peptides of the cancer antigen. It is therefore, highly desired to provide a vaccine preparation able to provide the cancer antigen in a vehicle in which is able to maximise the probability of presentation of the desired cancer antigen and which the number of possible competitive peptides for presentation are minimised. In this regard the molecules of the present invention have been analysed for the presence of peptides able to bind MHC class II molecules and in one embodiment of the invention such undesired peptide sequences with the ability to bind MHC class II have been altered such that the said binding interaction can no longer occur.

The ability of a peptide to bind a given MHC class II molecule for presentation on the surface of an APC is dependent on a number of factors most notably its primary sequence. This will influence both its propensity for proteolytic cleavage and also its affinity for binding within the peptide binding cleft of the MHC class II molecule. The MHC class II peptide complex on the APC surface presents a binding face to a particular-T cell receptor (TCR) able to recognise determinants provided both by exposed residues of the peptide and the MHC class II molecule. In the art there are procedures for identifying synthetic peptides able to bind MHC class II molecules, including for example methods for finding broadly reactive DR restricted epitopes [WO99/61916] however such peptides may not function as T cell epitopes in all situations particularly in vivo due to the processing pathways or other phenomena. Methods have also been provided to enable detection of T-cell epitopes by computational means scanning for recognised sequence motifs in experimentally determined T-cell epitopes or alternatively using computational techniques to predict MHC class II-binding peptides. One example is provided in WO02/069232 which teaches a computational threading approach to identifying polypeptide sequences with the potential to bind a sub-set of human MHC class II DR allotypes.

It is a particular objective of the present invention to provide polypeptide vaccine molecules in which the immune response to the vaccine is maximally focussed to a desired set of T-cell epitopes and the number of unwanted potential T-cell epitopes is reduced. It is possible to apply any of the methods disclosed previously [WO98159244; WO98/52976; WO00/34317, WO02/069232] to identify binding propensity of 708-derived peptides to an MHC class II molecule. In practice, the compositions embodied in the present invention have been derived following analysis conducted using a software tool exploiting the scheme outlined in WO02/069232. In brief, the software simulates the biological process of antigen presentation at the level of the peptide MHC class II binding interaction to provide a binding score for any given peptide sequence. Such a score is determined for many of the predominant MHC class II allotypes extant in the human population. As this scheme is able to test any protein sequence, the consequences of amino acid substitutions, additions or deletions with respect to the ability of a peptide to interact with a MHC class II binding groove can be predicted. Consequently new sequence compositions can be designed which contain reduced numbers of peptides able to interact with MHC class II and thereby function as immunogenic T-cell epitopes.

The process in arriving at the compositions disclosed herein therefore involved first identifying the presence of undesired MHC class II binding peptides, second, elimination of the undesired MHC class II binding sequence by amino acid substitution to render the sequence no longer able to bind with the MHC class II system and third, re-analysis of the modified sequence for any continued ability to bind to MHC class II molecules or for the presence of any further MHC class II ligands that may have been introduced during the modification.

MHC class II epitope removal has accordingly involved amino acid substitution to create modified variants depleted of undesired T-cell epitopes. The amino acid substitutions have been made at appropriate points within the peptide sequence predicted to achieve substantial reduction or elimination of the activity of the undesired T cell epitope. An “appropriate point” equates to an amino acid residue binding within one of the binding pockets provided within the MHC class II binding groove. It is most preferred to alter binding within the first pocket of the cleft at the so-called P1 or P1 anchor position of the peptide. The quality of binding interaction between the P1 anchor residue of the peptide and the first pocket of the MHC class II binding groove is recognised as being a major determinant of overall binding affinity for the whole peptide. An appropriate substitution at this position of the peptide will be for a residue less readily accommodated within the pocket, for example, substitution to a more hydrophilic residue. Amino acid residues in the peptide at positions equating to binding within other pocket regions within the MHC binding cleft are also considered and fall under the scope of the present.

As will be clear to the person skilled in art, multiple alternative sets of substitutions could be arrived at which achieve the objective of removing undesired epitopes. The resulting sequences would however remain broadly homologous with the specific compositions disclosed herein and therefore fall under the scope of the present invention. It would be typical to arrive at sequences that were around 70% or more homologous with the present specified sequences over their least homologous region and yet remain operationally equivalent. Such sequences would equally fall under the scope of the present.

The present invention discloses modified V-region sequences containing tracts of sequence which share homology with regions of the CEA molecule. Where such homologies exist, these are features intrinsic to the parental 708 antibody sequence. The invention provides for modified forms of the 708 parental sequence and in this regard provides sequences in which regions of the framework domains of the antibody contain residue substitutions for the purpose of eliminating or reducing unwanted immunogenic activity to the molecule on administration to the human subject. Unwanted immunogenic activity relates to sequence elements originating from the parental murine V-region and would not include sequence elements with homology to human CEA, human CD55 or elements of the 105AD7 antibody deliberately engineered into the sequence. The unwanted or non-desired epitopes as herein defined may be measured by the ability of the non-desired sequence element to bind to an MHC class II molecule or stimulate T-cells via presentation within an MHC class II molecule or bind to a soluble MHC class II complex which may bind to a human T cell or T-cell receptor complex.

Under the scheme of the present there are provided 4 different H-chain V-region sequences and 2 different L-chain V-region sequences. The present disclosure provides no limit to the possible combinations of H-chain and L-chain that may be provided to constitute a complete antibody molecule. Constitution of the complete antibody molecule may be achieved by recombinant DNA techniques and methods for purifying and manipulating antibody molecules well known in the art. Necessary techniques are explained fully in the literature, such as, “Molecular Cloning: A Laboratory Manual”, second edition (Sambrook et al., 1969); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds., 1987); “Current Protocols in Molecular Biology” (F. M. Ausubel et al., eds., 1987); “PCR: The polymerase Chain Reaction”, (Mullis et al., eds., 1994); “Current Protocols in Immunology” (J. E. Coligan et al., eds., 1991).

The preferred molecules of this invention can be prepared in any of several ways but is most preferably conducted exploiting routine recombinant methods. It is a relatively facile procedure to use the protein sequences and information provided herein to deduce a polynucleotide (DNA) encoding any of the preferred antibody V-regions. This can be achieved for example using computer software tools such as the DNSstar software suite [DNAstar Inc, Madison, Wis., USA] or similar. Any such DNA sequence with the capability of encoding the preferred polypeptides of the present or significant homologues thereof, should be considered as embodiments of this invention.

As a general scheme any of the VH or VL chain genes can be made using gene synthesis and cloned into a suitable expression vector. In turn the expression vector is introduced into a host cell and cells selected and cultured. The antibody molecules are readily purified from the culture medium and formulated into a vaccine preparation for therapeutic administration.

By way of a non-limiting example, one such scheme involves a gene synthesis process using panels of synthetic olignucleotides. The genes are assembled using a ligase chain reaction (LCR) wherein the oligonucleotides featuring complementary ends are allowed to anneal followed by amplification and fill-in using a polymerase chain reaction (PCR). The PCR is driven by addition of an increased concentration of the flanking oligonuclotides to act as primers. The PCR products are assembled into full-length antibody genes by further PCR from vectors containing 5′ and 3′ immunoglobulin gene flanking regions and sub-cloning into expression vectors for expression of whole antibody. The assembled VH and VL genes can serve as templates for mutagenesis and construction of multiple variant antibody sequences such as any of those disclosed herein. It is particularly conveinient to use the strategy of “overlap extention PCR” as described by Higuchi et al [Higuchi et al (1988) Nucleic Acids Res. 16: 7351] although other methodologies and systems could be readily applied.

Full-length immunoglobulin genes containing the variable region cassettes are assembled using overlapping PCR. Briefly, DNA of the vectors M13-VHPCR1 and M13-VKPCR1 [Orlandi et al (1989), PNAS, 89: 3833-7] are used as templates to produce a further two overlapping PCR fragments for each desired VH and VL chains including 5′ flanking sequence with the murine heavy chain immunoglobulin promoter and encoding the leader signal peptide and 3′ flanking sequence including a splice site and intron sequences. The DNA fragments so produced for each VH and VL are combined in a PCR using flanking primers required to obtain full-length DNA sequences.

The heavy chain gene complete with 5′ and 3′ flanking sequences are cloned into the expression vector pSVgpt [Reichmann et al (1988) Nature, 332: 323] which includes the human IgG1 constant region domain [Takahashi et al (1982) Cell, 29: 671] and the gpt gene for selection in mammalian cells. The light chain gene complete with 5′ and 3′ flanking sequences are cloned into the expression vector pSVHyg [Reichmann et al ibid] in which the gpt gene is replaced by the gene for hygromycin resistance (hyg) and includes a human kappa constant region domain [Heiter et al (1980) Cell, 22: 197]. For both vectors, the fully assembled VH or VL genes are sub-cloned as HindIII/BamHI fragments purified by gel electrophoresis and handled using well known procedures and reagent systems.

The heavy and light chain expression vectors are co-transfected using electroporation into NSO, a non-immunoglobulin producing mouse myeloma, obtained from the European Collection of Animal Cell Cultures (ECACC). Colonies expressing the gpt gene are selected in Dulbecco's Modified Eagles Medium (DMEM) supplemented with 10% (v/v) foetal calf serum and antibiotics (e.g. from Gibco, Paisley, UK) and with 0.8 μg/ml mycophenolic acid and 250 μg/ml xanthine (Sigma, Pople, UK). Production of human antibody by transfected cell clones is readily measured by ELISA for human IgG [Tempest et al (1991) Bio Technology 9: 266]. Cell lines secreting antibody are expanded and antibody purified by protein A affinity chromatography [Harlow E. & Land D.; bid]. The concentration of the purified antibody is determined using an ELISA detecting the human kappa constant region of the antibodies of interest

The molecules according to the invention may be administered alone in a monotherapy or in combination with other pharmaceutically effective drugs. Such drugs may include immunotherapeutic agents or chemotherapeutic agents which contain cytotoxic effectve radio labeled isotopes, or other cytotoxic agents, such as a cytotoxic peptides (e.g. cytokines) or cytotoxic drugs and the like. The term “cytotoxic agent” as used herein refers to a substance that inhibits or prevents the function of cells and/or causes destruction of cells. The term is intended to is include radioactive isotopes, chemotherapeutic agents, and toxins such as enzymatically active toxins of bacterial, fungal, plant or animal origin, or fragments thereof. The term may include also members of the cytokine family, preferably IFNγ as well as anti-neoplastic agents having also cytotoxic activity. The term “chemotherapeutic agent” or “anti-neoplastic agent” is regarded according to the understanding of this invention as a member of the class of “cytotoxic agents”, as specified above, and includes chemical agents that exert anti-neoplastic effects, i.e., prevent the development, maturation, or spread of neoplastic cells, directly on the tumor cell, e.g., by cytostatic or cytotoxic effects, and not indirectly through mechanisms such as biological response modification. Suitable chemotherapeutic agents according to the invention are preferably natural or synthetic chemical compounds, but biological molecules, such as proteins, polypeptides etc. are not expressively excluded. There are large numbers of anti-neoplastic agents available in commercial use, in clinical evaluation and in pre-clinical development, which could be included in the present invention for treatment of tumors/neoplasia by combination therapy with TNFα and the anti-angiogenic agents as cited above, optionally with other agents such as EGF receptor antagonists. It should be pointed out that the chemotherapeutic agents can be administered optionally together with above-said drug combination. Examples of chemotherapeutic or agents include alkylating agents, for example, nitrogen mustards, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; cytotoxic antibiotics and camptothecin derivatives. Preferred chemotherapeutic agents or chemotherapy include amifostine (ethyol), cisplatin, dacarbazine (DTIC), dactinomycin, mechlorethamine (nitrogen mustard), streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, eniposide, testolastone, thioguanine, thiotepa, uracil mustard, vinorelbine, chlorambucil and combinations thereof. Most preferred chemotherapeutic agents according to the invention are cisplatin, gemcitabine, doxorubicin, paclitaxel (taxol) and bleomycin.

It is a further aspect of the present invention that it relates to methods for therapeutic treatment of humans using the modified antibody compositions. For administration to an individual, any of the modified antibody compositions would be produced to be preferably at least 80% pure and free of pyrogens and other contaminants. It is further understood that the therapeutic compositions of the modified antibody proteins may be used in conjunction with adjuvants and carrier substances commonly known in the art. Such substances in themselves provide no immunogenic epitopes. A well known adjuvant comprises a mineral oil emulsion and is termed Freunds adjuvant but other preparation may equally be considered for example EP-A-0745388, EP-A-0781559, U.S. Pat. Nos. 5,057,540; 5,407,684; 5,077,284; 4,436,728; 5,171,568; and U.S. Pat. No. 4,726,947 or similar. The vaccine preparation will preferably be administered with a pharmaceutically acceptable excipient. Such excipients can act as a diluent but can include stabilising agents, wetting and, emulsifying agents, salts, encapsulating agents, buffers, and skin penetration enhancers. Examples are described in Remington's Pharmaceutical Sciences (Alfonso R. Gennaro, ed., 18th edition, 1990). Lipospmre encapsulation may also be considered as a means for formulating the proteins for therapeutic use and such use may also include therapeutic schemes involving biological response modifiers such as GM-CSF and or IL-2 or other proteins.

It is recognised that to elicit an immune response or treat an individual for a CEA-associated tumour, the vaccine preparation is administered to an individual parenterally, and could include intracutaneous, intramuscular or intradermal administration. The terms “cancer” and “tumour” refer to or describe the physiological condition in mammals that is typically characterized by unregulated cell growth. By means of the molecules and pharmaceutical compositions according of the present invention tumors, preferably CEA-associated tumours, can be treated such as tumors of the breast, heart, lung, small intestine, colon, spleen, kidney, bladder, head and neck, ovary, prostate, brain, pancreas, skin, bone, bone marrow, blood, thymus, uterus, testicles, cervix, and, liver. Preferred cancers according to the invention are colorectal, gastric, pancreatic, non-small cell lung and breast cancers.

The molecules according to the invention are administered to an individual by means of a pharmaceutical composition. The “pharmaceutical compositions” of the invention can comprise agents that reduce or avoid side effects associated with the combination therapy of the present invention (“adjunctive therapy”), including, but not limited to, those agents, for example, that reduce the toxic effect of anticancer drugs, e.g., bone resorption inhibitors, cardioprotective agents. Said adjunctive agents prevent or reduce the incidence of nausea and vomiting associated with chemotherapy, radiotherapy or operation, or reduce the incidence of infection associated with the administration of myelosuppressive anticancer drugs. Adjunctive agents are well known in the art.

The immunoglobulin agents according to the invention are preferably administered in combination with adjuvants like BCG and immune system stimulators.

The amount of vaccine preparation to be administered depends upon several factors, for example the condition of the patient and route of administration. A non-limiting example dosage regime would range from about 0.1 mg to about 20 mg and the dosing regimen could be bi-weekly for four injections, followed by monthly injections as required. Maintenance doses will depend, on the condition and response of the individual being treated.

The vaccine preparations of the invention are considered particularly useful as a therapeutic adjunct to conventional surgical intervention for cancer and therefore will serve to reduce the likelihood of tumour recurrence and clinical relapse.

The effectiveness of the vaccine administration would include clinical tests to determining the progression of cancer for example detection of inflammatory indicators, mammography, radioscintigraphy and any of the other clinical investigations well known in the art.

It is particularly preferred to determine the cellular immune response in a patient receiving the vaccines of the present invention. Especially preferred assays focus on specific T cell activity and would include for example measurement of T cell proliferation. In this assay, peripheral blood mononuclear cells (PBMC) are obtained from a whole blood sample. The cells are cultured in the presence of synthetic peptides such as derived from human CEA or human CD55 or alternatively challenged with whole CEA protein or irradiated CEA expressing cells at various concentrations. Preferably, the stimulator cells are autologous with the responder cells. A stimulation index (SI) is determined typically using ³H-thymidine incorporation as a marker of cellular proliferation. In such an assay the positive CEA or CD55 induced proliferation would be concluded if the measured SI was at least at a value of 2.0 preferably 2.5 or greater. For this assay, the SI=CPM test culture/CPM untreated control culture.

Stimulation of Th1 T-cells, which provide “help” to the formation of cytotoxic T-cells, can be measured by assay of the production of interferon-gamma in the culture supernatant at day 8-10. Interferon-gamma production is readily measured using commercially available ELISA based systems.

Activity of CEA or CD55 specific cytoxic T cells would also be particularly informative. Particularly suitable assays of this type are described both by Kantor et al [Kantor, J. et al (1992) Cancer Res. 52: 6917-6925 & JNCI (1992) 84:1084-1092] and Kwong et al [Kwong, Y. et al (1995) JNCI 87: 982-990]. The assay involves measurement of ⁵¹Cr release into the medium from labelled CEA expressing target cells and the percent specific release of ⁵¹Cr into the medium is measured in comparison with labelled targets cultured alone (negative control) and target lysed with a detergent (positive control).

By way of a further non-limiting example, the method for the derivation of the preferred protein sequences of the invention is now described: The production of chimaeric 708 has been described previously (Durrant, L. G., et al (2001), lnt. J. Cancer. 92:414-420). The variable region protein sequences were examined for the presence of un-wanted T-cell epitopes using methods described in WO98/52976 and sequence variants designed. Additional analysis was conducted on the human CEA protein sequence [Schrewe, H. et al (1990), Mol. Cell. Biol. 10: 2738-2748], the human CD55 protein sequence [Caras, I. W. et al (1987) Nature 325: 545-549] and the antibody 107AD5 variable region sequences [WO97/32021]. Analysis comprised homology alignments using commercially available software suites (e.g. “DNAstar”, DNASTAR Inc, Madison, Wis., USA) and epitope analysis as described elsewhere [WO98/59244; WO98/52976; WO00/34317; Rammensee, H. G. et al (1999) ibid]. The scheme for the analysis of pepuide sequences with potential to act as MHC class II binding ligands has been described in detail previously [WO 02/069232]. Using this procedure, multiple MHC class II ligands for one or more allotypes have been identified in the antibody 708 V-region domains. Variant sequences were compiled which were depleted of MHC class II ligands. This was achieved by iterative cycles of amino acid substitution and re-analysis to confirm epitope removal. The protein sequences of the desired compositions are shown in FIGS. 4-9.

1. An immunoglobulin molecule or a fragment thereof deriving from a parental anti-idiotype anti-CEA antibody and comprising constant regions from human origin and synthetically designed variable regions comprising one or more sequence tracts of more than 4 consecutive amino acid residues deriving from human tumor antigen CEA (carcinoembryonic antigen; SEQ ID NO: 3).

2. An immunoglobulin molecule according to claim 1, wherein one of said sequence tracts comprises 5-20 consecutive amino acid residues.

3. An immunoglobulin molecule according to claim 1, wherein at least one of said sequence tracts is a component of a complementarity determining region (CDR) of the heavy and/or light chain of said immunoglobulin or overlaps with adjacent residues of a framework region adjacent to said CDR.

4. An immunoglobulin molecule of claim 3, wherein said component forms 30 to 100% of the amino acid residues of said CDR.

5. An immunoglobulin molecule according to claim 3, wherein said CDR is a CDR of the heavy chain of said immunoglobulin.

6. An immunoglobulin molecule according claim 3, wherein at least two CDRs of each heavy and light chain consist completely of CEA-derived sequence tracts.

7. An immunoglobulin molecule according to claim 1, wherein said parental anti-idiotype anti-CEA antibody is mouse antibody 708.

8. An immunoglobulin molecule according to claim 1, comprising within the variable regions additionally sequence tracts of 5 to 25 consecutive amino acid residues deriving from human CD55 (SEQ ID NO: 4) antigen or the hypervariable regions of an anti-idotype anti-CD55 antibody.

9. An immunoglobulin molecule of claim 8, wherein said anti-idotype anti-CD55 antibody is mouse antibody 105AD7.

10. An immunoglobulin molecule according to claim 1, wherein within the variable regions additionally potential MHC class II epitopes, which do not contribute to an immune response to CEA positive human cancer cells, have been removed by amino acid substitutions.

11. An immunoglobulin molecule according to claim 1, comprising within the variable regions additionally CEA derived sequence tracts from SEQ ID NO: 3 which are MHC class I epitopes.

12. An immunoglobulin molecule according to claim 11, wherein said CEA-derived sequence tracts are TLLSVTRNDV (SEQ IS NO: 7) and YLSGANLNL (SEQ IS NO: 8).

13. An immunoglobulin molecule of claim 11, wherein said CEA derived sequence tracts are part of or form completely one ore more of the CDRs of the light chain of said immunoglobulin.

14. An immunoglobulin molecule according to claim 1, comprising within the variable regions additionally CEA derived sequence tracts from SEQ ID NO: 3 which are MHC class II epitopes contribute to an immune response directed to CEA positive human cancer cells.

15. An immunoglobulin molecule according to claim 1, comprising a variable heavy chain selected from any of the sequences as depicted in FIGS. 4 to 7.

16. An immunoglobulin molecule according to any of the claim 1, comprising a variable light chain selected from any of the sequences as depicted in FIGS. 8 and 9.

17. An immunoglobulin molecule according to claim 1, comprising a heavy chain selected from any of the sequences as depicted in FIGS. 4 to 7 and a light chain selected from any of the sequences as depicted in FIGS. 8 and 9.

18. An immunoglobulin molecule according to claim 1, wherein the variable heavy and/or light chain comprises one or more sequence tracts in identity with the sequence tracts selected from the group:
(i) 345-354 of SEQ ID NO: 3;
(ii) 387-396 of SEQ ID NO: 3;
(iii) 571-579 of SEQ ID NO: 3;
(iv) 629-645 of SEQ ID NO: 3;
(v) 148-167 of SEQ ID NO: 4.

19. A pharmaceutical composition comprising an immunoglobulin molecule of claim 1 in an biologically effective amount, an adjuvant, and optionally a pharmaceutically acceptable carrier, diluent or excipient.

20-21. (canceled)

22. A method for the production of a vaccine molecule based on a synthetically designed immunoglobulin molecule suitable for the treatment of a human individual suffering from a CEA (carcinoembryonic antigen) positive solid or metastasising tumor, said method comprising the following steps:
(i) selecting a non-human anti-idiotype anti-CEA antibody,
(ii) replacing the non-human constant regions by human constant regions, and
(iii) replacing partially or completely one or more of the hypervariable regions (CDRs), with sequence tracts deriving from CEA (SEQ ID NO: 3), whereby optionally, framework residues adjacent to said CDRs are included.

23. The method of claim 22, comprising additionally one or more of the steps selected from the group consisting of:
(iv) replacing sequence tracts within the variable regions with tracts deriving from CD55 antigen (SEQ ID NO: 4) or the hypervariable regions of an anti-idiotype anti-CD55 antibody,
(v) replacing sequence tracts within the variable regions with tracts which are MHC class I and/or MHC class II epitopes responding to CEA positive human cancer cells, and
(vi) removing within the variable regions potential MHC class II epitopes, which do not contribute to an immune response to CEA positive human cancer cells.

24. The method of claim 22, wherein said non-human anti-idiotype anti-CEA antibody is mouse antibody 708.

25. A method of treating a tumor in a human patient comprising administering an anti-tumor effective amount of an immunoglobulin molecule of claim 1 to a human patient having a solid or metastasizing tumor.

26. A method of stimulating T-cells against a tumor in a human patient comprising administering a T-cell stimulating amount of an immunoglobulin molecule of claim 1 to a human patient having a solid or metastasizing tumor.
